(19) United States
(12) Patent Application Publication
(10) Pub. No.: US 2023/0001411 A1
(43) Pub. Date: Jan. 5, 2023

(54) Portable device for in situ genetic analyses

(71) Applicant: Enbiotech S.r.I., Palermo PA (IT)

(72) Inventors: Cosimo MANZO, Palermo PA (IT); Guido SPOTO, Palermo PA (IT)

(21) Appl. No.: 17/772,555

(22) PCT Filed: Oct. 29, 2020
(86) PCT No.: PCT/IB2020/060136

§ 371 Date: Apr. 28, 2022

(30) Foreign Application Priority Data
Oct. 29, 2019 (IT) 102019000019942

(51) Int. Cl. B01L 3/00; C12Q 1/6841

(52) U.S. Cl. CPC B01L 3/50853; C12Q 1/6841; B01L 2200/18; B01L 2300/025; B01L 2300/021; B01L 2200/16; B01L 2300/0654; B01L 2300/0829

(57) ABSTRACT

A device for performing in situ genetic analyses, conformed so as to be transportable manually by a user, which comprises a casing defining an internal compartment and a plurality of analysis units arranged in the internal compartment, where each analysis unit is configured to perform a respective and independent genetic analysis of at least one sample; each analysis unit comprises a sample holder compartment accessible by the user and adapted to accommodate at least one sample; a command and control unit; at least one sensor selected among an optical, acceleration, temperature, pressure, motion, chemical sensor or a combination thereof, configured to detect a first physical quantity relative to the genetic analysis of the at least one sample and to transduce the first physical quantity into a first signal which is indicative of the state of progress of the genetic analysis, the command and control unit is in signal communication with the at least one sensor for receiving said first signal; the analysis unit further comprises a plurality of instruments, configured to perform the genetic analysis of the sample, comprising an amplification and optical detection device configured to detect at least a second physical quantity relative to the genetic analysis and to transduce the second physical quantity into at least a second signal which is indicative of the outcome of the genetic analysis, the command and control unit is in signal communication with the amplification and optical detection device for receiving said second signal; the device further comprises a processing unit, in signal communication with the command and control unit of each analysis unit for receiving the respective first signals and the respective second signals.

### FIELD OF APPLICATION

The present description relates to a device, a system and an assembly for performing field genetic analyses, and the related method of use.

In particular, the present description refers to a device, a system and an assembly which allow a non-specialized operator to be able to perform field genetic analyses.

### DESCRIPTION OF THE PRIOR ART

It is known in the state of the art to use devices which allow a specialized operator, such as a biotechnologist, to be able to carry out genetic analyses in a specialized laboratory. In the field of molecular biology, genetic analyses are carried out in specialized laboratories to allow qualified operators to be able to safely perform all the procedures for analysing genetic material (DNA and RNA).

Genetic analyses, carried out with molecular biology techniques, are increasingly used in various fields and sectors, such as in the agri-food sector for verifying the varietal authenticity of plants and species of animal raw materials used for the production of finished products. Genetic analyses are also used in the health sector for the diagnosis of genetically determined diseases, and in the veterinary field for the detection of microorganisms responsible for pathologies in animals and for the recognition of animal species.

It should be specified that the procedures in use, through which it is possible to perform genetic analyses, are characterized by the execution of mandatory operations. In particular, all the genetic analysis procedures known in the state of the art include three mandatory steps: a) a first step of extracting the nucleic acid (DNA or RNA) from a sample, b) a second step of amplifying the nucleic acid extract, c) a third step of detecting and interpreting the result. It should be noted that all the devices necessary to perform genetic analyses are present in any specialized laboratory, whereas the necessary reagents and materials are available on the market, sometimes integrated and proposed in the form of laboratory kits for the performance of a specific genetic analysis. By way of example, think of the kit for the performance of a genetic analysis to determine a pathology.

In detail, step a) of extracting the nucleic acid is performed by using reagents that need to be stored at a controlled temperature (for example minus twenty degrees centigrade or four degrees centigrade), or alternatively that must be ready for use and stable at room temperature. In the latter case, the operations of extracting the genetic material take only a few minutes and are considerably simplified. The extraction systems are also available on the market in special kits for genetic analyses, both in the human and in the agri-food sector. In the state of the art, kits for the genetic analyses provide for the nucleic acid to be extracted in the laboratory, by qualified staff and with the aid of devices such as for example micropipettes, benchtop centrifuges and vortexes. The known kits comprise the reagents necessary to perform step a) of extracting the nucleic acid, such reagents comprise for example solutions for the cell lysis of the sample by chemical reaction.

Step b) of amplifying the nucleic acid is carried out by means of the polymerase chain reaction technique, also known as PCR, which allows a specific genetic sequence to be replicated in vitro in order to obtain a sufficient quantity of genetic material for detection. The known kits comprise reagents, such as a solution composed of taq polymerase (enzyme), buffer, nucleotides and magnesium chloride. Said reagents are required to let the gene amplification reaction take place by means of PCR. Furthermore, kits are known which comprise reagents for performing step b) of amplifying the nucleic acid by using reagents and materials ready for use and stored in a stable manner at room temperature, for example lyophilized and vacuum-packed. Step b) of amplifying the nucleic acid must also take place in the laboratory, by qualified staff and with the aid of devices such as micropipettes, mini-centrifuges, thermocyclers and vortexes.

The step c) of detecting and interpreting the result provides that the detection of the result can be performed in different ways, for example through horizontal electrophoresis on agarose gel or, alternatively, by detecting the fluorescence emitted by the analysed sample. In the first case, the known kits comprise for example the precast agarose gel, the electrophoretic miming buffer pre-dosed in a pouch, the molecular weight marker, and the interpretation of the results is entrusted to the operator. The detection takes place in the laboratory, by qualified staff and with the aid of micropipettes, cells and power supplies for electrophoresis, ultraviolet gel readers. As an alternative to ultraviolet gel readers, for example a fluorescence stress and detection system can be used. In this case, known kits provide for the detection to take place through a fluorescence reader, which is also responsible for the automatic interpretation of the results.

Miniaturized laboratories are known in the state of the art which use a device in which, thanks to microfluidic systems (also called lab-on-chip), particularly rapid genetic analyses are carried out. In particular, such microfluidic devices are able to perform steps a) of extracting the nucleic acid and b) of amplifying the nucleic acid in a semi-automatic and simplified manner. That is to say that a specialized operator inserts the materials necessary for the genetic analysis into the device and obtains as a result the amplified DNA on which to perform the step c) of detection and interpretation. However, it should be pointed out that for said genetic analysis to be performed it is necessary that the microfluidic device is used in the laboratory and in combination with instruments such as pumps, syringes, thermocyclers, microscopes and fluorescence readers. Also in this case, genetic testing must be performed by a specialised operator in a suitable laboratory or facility.

Automatic devices called PCR workstations are also known in the state of the art. These are open work platforms containing part of the instrumentation and the consumables necessary to perform genetic tests, structured in a modular way according to the operator's needs. These PCR workstations work through an automatic dispensing system that distributes the reagents, used to carry out the genetic analyses, into special tubes in the appropriate quantities. The PCR workstations are also equipped with a software, which can be installed in an external PC or on a special device (for example a touch screen control panel) that guides the operator in the performance of the various operating steps and allows the operating status of the instrumentation and any graphs obtained at the end of the genetic test to be displayed. Inside the workstation there are also optical sensors that check the work surface before carrying out the various operations by evaluating the presence of consumables and the liquid level of the reagents. The workstations also comprise a barcode reader that recognizes the consumables used by the operator (e.g., tubes, plates, tips) and the samples analysed. The workstation may also comprise accessories such as a thermoblock that allows the sample or reagents to be cooled and heated (between 0-90° C.) and a vessel to eliminate separately solid and liquid waste. Inside the workstation it is possible to automatically carry out all the operations necessary to perform a genetic test, from the extraction of the nucleic acid to the detection of the final result.

Documents US 2016/054343 A1 and US 2013/115607 A1 describe systems and methods for carrying out genetic analyses on a sample through a device configured to carry out various operations, including receiving the sample, preparing the same sample and carrying out the analysis thereof. This device can be designed to accommodate more than one sample in order to carry out various analyses simultaneously.

### Problem of the Prior Art

Genetic analyses are currently performed exclusively in specialized laboratories, equipped with highly qualified equipment and professionalism, to which the samples are to be sent in order to be analysed. The costs of the analyses and the waiting times for the results, aggravated by the shipping times of the samples, are in some cases incompatible with the needs of industries (for example the agri-food industry) which require speed and simplicity of execution. Therefore, the companies cannot carry out field genetic analyses, and it is not possible to obtain results in a short time.

As mentioned above, there are also miniaturized systems, that is microfluidic systems, which thanks to the small size and the multiplicity of functions that they can perform on a single chip, are currently identified as miniaturized laboratories. It should be noted that microfluidic systems can only be used by specialized staff in an environment suitable for performing genetic analyses. Furthermore, microfluidic systems are small-sized devices and therefore easy to transport, but for them to be used for genetic analyses they need to operate in combination with instruments that are normally present in a specialized laboratory only.

As mentioned above, there are also PCR workstations, for which it should be noted that although they make all the operations necessary for performing a genetic test fully automatic and controlled, they are very cumbersome and are not suitable for carrying out in situ tests, since they require an organized facility, such as specialized laboratories, and large working spaces. It should be noted that this type of platform requires the presence of a specialized operator for interpreting the results and for selecting the type of operations required for the performance of a genetic test.

In summary, the known systems presuppose the use by operators with in-depth knowledge of the devices and of the common molecular biology operations, as well as in-depth knowledge of the steps of nucleic acid extraction, of amplification of the extracted nucleic acid and of detection and interpretation of the result.

Therefore, known systems can only be used by specialized staff inside suitable laboratories. In fact, the skills of a specialized operator are demanded in order to have a correct interpretation and validation of the results. Such systems are complex and require long usage times. Therefore, known systems do not allow to perform in situ genetic analyses.

### SUMMARY OF THE INVENTION

The object of the present invention is to realise a portable device complete with all the necessary instrumentation in order to be able to perform genetic analyses outside a suitable facility, such as for example a specialized laboratory.

A further object of the present invention is to realise a system capable of guiding and monitoring an operator during the performance of all the steps necessary for a genetic analysis.

A further object of the present invention is therefore to realise a system capable of providing an automatic detection and interpretation of the result.

A further object of the present invention is to realise a system equipped with mechanisms for the automatic check of the correct performance of the activities carried out by the operator for the automatic validation of the result.

A further object of the present invention is to realise an assembly in order to be able to perform genetic analyses together with the reagents, too.

A further object of the present invention is to realise a method for performing a genetic analysis by using an assembly for genetic analyses within the scope of the present invention.

### Advantages of the Invention

Thanks to an embodiment, it is possible to realise a device for genetic analyses that can be used in situ in order to be able to perform field genetic analyses in a short time.

Thanks to a further embodiment, it is possible to realise a system for genetic analyses which allows a non-specialized operator to be able to perform genetic analyses autonomously and safely.

Thanks to a further embodiment, it is possible to realise a method for using an assembly for genetic analyses of the present invention, which allows a non-specialized operator to be able to autonomously perform field genetic analyses.

### BRIEF DESCRIPTION OF THE DRAWINGS

The characteristics and advantages of the present disclosure will become clear from the following detailed description of a possible practical embodiment, illustrated by way of non-limiting example in the set of drawings, wherein:

FIG. 1 shows a schematic representation of a device inserted in a system according to the present invention;

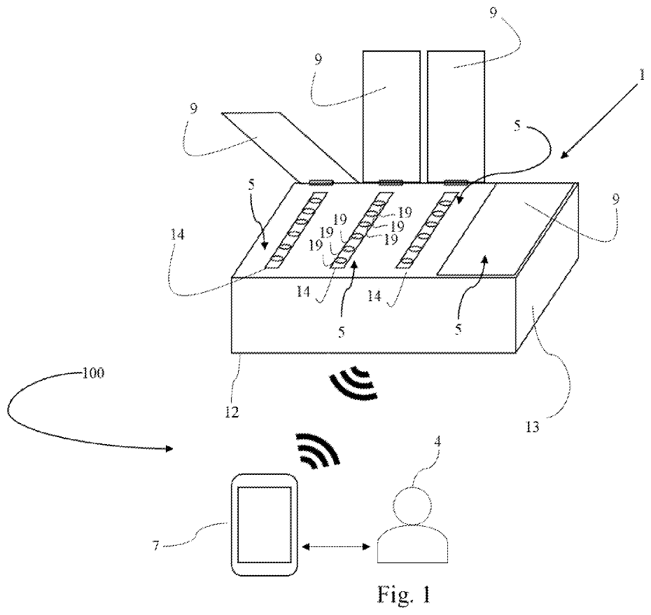

FIG. 2 shows a schematic representation of an embodiment of a system according to the present invention;

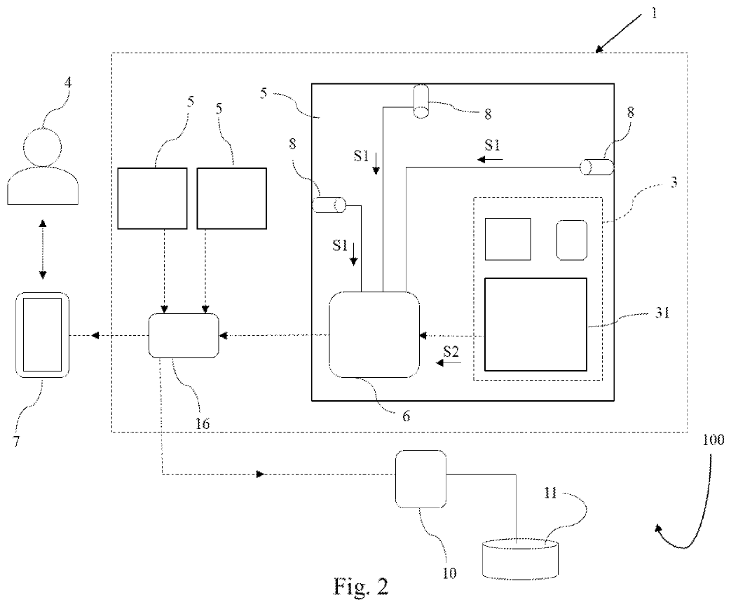

FIG. 3 shows a schematic representation of an embodiment of an assembly according to the present invention.

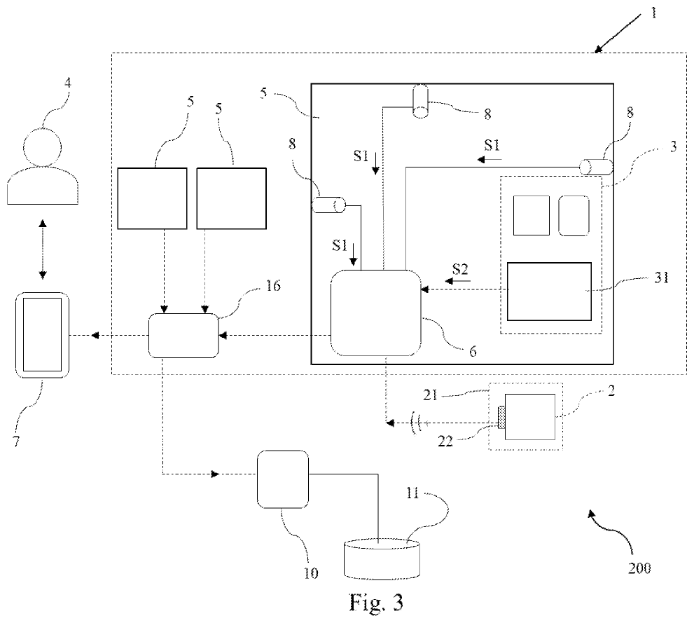

### DETAILED DESCRIPTION

Even when not explicitly highlighted, the individual features described with reference to the specific embodiments must be considered as accessories and/or exchangeable with other features, described with reference to other embodiments.

The present invention relates to a device 1 for performing genetic analyses conformed so as to be transportable manually by a user 4. Advantageously, the user 4 can carry out the in situ genetic analysis. In the following, for brevity's sake, reference will be made in a non-limiting way to an in situ genetic analysis as a genetic analysis carried out in the field, that is to say in the specific place where it is intended to carry out a genetic analysis in a rapid and timely manner, and outside a specialized laboratory. A place where it is possible to carry out field genetic analysis is for example the building of an agri-food industry, a medical-veterinary clinic, customs offices or a pharmacy. The device 1 can be contained in a suitcase or bag or similar container to allow the user 4 to be able to transport the entire device 1 to the place where a genetic analysis is to be carried out. Advantageously, the user 4 can transport the device 1 to an agricultural site, a company, a medical or veterinary surgery and in general to the place where an in situ genetic analysis is to be performed.

The device 1 comprises a casing 12 defining an internal compartment 13. The device 1 comprises at least one analysis unit 5 housed in the internal compartment 13. The analysis unit 5 is configured to perform a genetic analysis of at least one sample and comprises a sample holder compartment 14 which is accessible by the user 4 and adapted to accommodate the at least one sample. Preferably, the sample holder compartment 14 comprises a plurality of seats 19 each of them configured to house a respective sample. Preferably, the sample holder compartment 14 extends longitudinally defining a groove in which several seats are aligned, preferably twelve seats 19. Advantageously, it is possible to carry out the genetic analysis of several samples simultaneously in order to have a reliable result.

The device 1 comprises a plurality of analysis units 5 arranged in the internal compartment 13. Each analysis unit 5 is configured to perform a respective and independent genetic analysis.

In the following, for brevity's sake, reference will be made in a non-limiting way to a genetic analysis as the procedure through which it is possible to obtain a genetic identification of at least one biological sample to be analysed. In particular, reference will be made to a plurality of steps necessary for the performance of a genetic analysis as the set of all the passages that are necessary for preparing and genetically analysing at least one sample. The genetic analysis is conducted through a plurality of steps, among which three are mandatory for each type of genetic analysis: a) a first step of extracting the nucleic acid (DNA or RNA) from a sample, b) a second step of amplifying the extracted nucleic acid, c) a third step of detecting and interpretation the result.

Preferably, the technology used to perform step b) of amplifying the nucleic acid preferably comprises the LAMP (Loop Mediated Isothermal Amplification) technology.

The LAMP technology is a type of DNA amplification that allows obtaining numerous copies of DNA using a single reaction temperature, thanks to the use of a specific enzyme (i.e., Bst polymerase). Compared to traditional gene amplification techniques, LAMP is much faster and easier to perform, besides having lower costs for both reagents and instrumentation necessary for the analyses. This technology is suitable for performing genetic analyses quickly, without using particular laboratory equipment and with no need for specialized staff.

Each analysis unit 5 comprises a command and control unit 6.

The analysis unit 5 comprises at least one sensor 8. Each sensor 8 is in signal communication with the command and control unit 6. The sensor 8 is selected among an optical, acceleration, temperature, pressure, motion, chemical sensor or a combination thereof. In particular, the sensor 8 is configured to detect a first physical quantity relative to the genetic analysis of the sample and to transduce said first physical quantity into a first signal S1. The first signal S1 is indicative of the state of progress of the genetic analysis.

In other words, each sensor 8 is capable of monitoring a respective first physical quantity associated with the genetic analysis, in order to provide a live monitoring of the genetic analysis being executed. By way of example, think of an optical sensor capable of detecting the level of the solution in a test tube, or a temperature sensor for monitoring the temperature in the analysis unit 5 or a chemical sensor for detecting the pH of a solution. In this way it is possible to monitor physical quantities relative to the various steps of the genetic analysis so as to detect any errors made by the user 4.

The command and control unit 6 is configured to receive and process the first signal S1.

Each analysis unit 5 comprises a plurality of instruments 3 configured to perform the genetic analysis of the at least one sample using reagents. In the following, for brevity's sake, reference will be made in a non-limiting way to a plurality of instruments 3 as the known scientific instrumentation, laboratory or portable, specifically developed for performing the plurality of steps of the genetic analysis. Preferably, the plurality of devices 3 comprises, for example, one or more of micropipettes, thermocyclers, fluorescence detectors.

In particular, for the LAMP technology to be implemented, the plurality of instruments 3 comprises an amplification and optical detection device 31.

In a preferred embodiment, the amplification and optical detection device 31 is configured to be able to perform step b) of amplifying the extracted nucleic acid and step c) of detecting and interpreting the result of the genetic analysis.

This amplification and optical detection device 31 is in signal communication with the respective command and control unit 6. The amplification and optical detection device 31 is configured to detect at least a second physical quantity relative to the genetic analysis and to transduce said second physical quantity into at least a second signal S2 which is indicative of the outcome of the genetic analysis.

Preferably, the amplification and optical detection device 31 comprises a heating system for heating, to the amplification temperature, an optical and optoelectronic system comprising optical filters and an array of LEDs and an array of photodiodes for stressing and detecting the fluorescence of the samples to be analysed. In particular, the amplification and optical detection device 31 is capable of detecting at least two different pairs of excitation/emission wavelengths, in order to be able to detect the presence of the genetic sequence to be identified through genetic analysis. Therefore, as a function of the detected wavelength values (i.e., second physical quantity), the amplification and optical detection device 31 generates the at least one second signal S2.

The command and control unit 6 is configured to receive and process at least a second signal S2.

The device 1 comprises a processing unit 16, in signal communication with the command and control unit 6 of each analysis unit 5. The processing unit 16 is configured to receive and process the respective first signals S1 and the respective second signals S2.

The analysis units 5 are arranged in an array in the internal compartment 13. Preferably, the analysis units 5 are identical to each other and have an elongated shape. Preferably, each analysis unit 5 is contained in a parallelepiped-shaped block. Advantageously, it is possible to arrange them in the device 1 in a compact manner. Preferably, the analysis units 5 are contained in the internal compartment 13 one next to the other forming a single block.

According to a preferred embodiment, the device 1 comprises four analysis units 5. Advantageously, the device 1 can perform up to four independent genetic analyses.

According to a preferred embodiment, each analysis unit 5 comprises a door 9 for covering the sample holder compartment 14. This door is swinging and is hinged to the device 1. In particular, each door 9 can be opened to allow the user 4 to access the sample holder compartment 14 to carry out operations on the sample to be analysed. In this way it is possible to access an analysis unit 5 and the respective sample holder compartment 14 if necessary, avoiding contamination of the samples during the genetic analysis. Advantageously, the sample holder compartment 14 is able to provide the user 4 with a work environment suitable for performing the genetic analysis. Advantageously, it is also possible to start different analyses in the respective analysis units 5 at different times.

In accordance with a preferred embodiment, the processing unit 16 is configured to automatically identify the operator 4 by means of identification codes, badges or radio frequency bracelets. In this way, only authorized operators are able to use the device 1.

In accordance with a preferred embodiment, the device 1 comprises an electric mains or autonomous power supply system (e.g., power supply via rechargeable batteries or via a solar power system). Preferably, the power supply system is capable of powering each electronic device present inside the device 1.

The present invention also relates to a system 100 for performing in situ genetic analyses. The system 100 comprises the device 1. The system 100 further comprises a graphic display interface 7. Said graphic display interface 7 is in signal communication with the processing unit 16.

According to a preferred embodiment, the processing unit 16 comprises the graphic display interface 7. In accordance with a preferred embodiment, the processing unit 16 comprises an electronic board capable of managing any peripherals, communication via Bluetooth and/or Wi-Fi and can be equipped with RAM, USB ports, LAN ports, 32 or 64 bit microprocessor, expansion port via SD card and/or voltage regulation system. Preferably, the graphic display interface 7 comprises a PC tablet, a smartphone or a PC in signal communication, via cable or Wi-Fi, with the processing unit 16.

The processing unit 16 is configured to display a first message as a function of each first signal S1 through the graphic display interface 7.

In particular, the processing unit 16 is configured to generate, through the graphic display interface 7 and as a function of the value of S1, a first message in order to carry on with the execution of the plurality of steps. The command and control unit 6 processes the first signal S1 received by a respective sensor 8 so as to identify any execution errors made by the user 4 during the execution of the plurality of steps of the genetic analysis. Then the processing unit 16 receives the first signal S1 and sends a first message to the graphic display interface 7.

Therefore, a first message is displayed on the graphic display interface 7 to authorize the user 4 to carry on with the genetic analysis, continuing in the plurality of steps or repeating the steps compromised by a possible error made by the user 4. Advantageously, the processing unit 16 is able to monitor the user 4 during the execution of the plurality of steps of the genetic analysis through the at least one sensor 8, so as to identify any errors made by the user 4. The processing unit 16, also through the graphic display interface 7, is able to clearly and precisely indicate to the user 4 the subsequent step of the genetic analysis that he will have to carry out. In this way, it is possible to allow a non-specialized user 4 to be able to perform a genetic analysis outside a certified laboratory, with total safety and reliability.

The processing unit 16 is also configured to generate a second message through the graphic display interface 7 and as a function of the value of the second signal S2. The second message contains data relative to the results, either positive or negative, of the genetic analysis.

In accordance with a preferred embodiment, the amplification and optical detection device 31 is configured to recognize if the at least one second signal S2 is free from errors deriving from an incorrect performance of the genetic analysis by the user 4.

In accordance with a preferred embodiment, the processing unit 16 is in signal communication with a remote control unit 10 positioned externally to the system 1 to transmit the first signal S1 and/or the second signal S2. Preferably, the remote control unit 10 is configured to receive and store the first signal S1 and/or the second signal S2 in a database 11 associated therewith.

Advantageously, the remote control unit 10 stores in the database 11 all the data relative to the genetic analysis and in particular data relative to the signals detected by the sensors 8 arranged in the analysis unit of the system 100 and to the result of the genetic analysis performed.

In accordance with a preferred embodiment, each sample to be analysed comprises an RFID tag containing data relative to the sample, and the processing unit 16 is configured to read the data relative to the sample in order to display a third message through the graphic display interface 7. The third message preferably comprises information relative to the sample.

The present disclosure also relates to an assembly 200 for performing in situ genetic analyses. Said assembly 200 comprises a system 100 and in addition a kit 2 for carrying out at least one genetic analysis of at least one sample. In particular, said kit 2 comprises laboratory instrumentation and reagents for carrying out a specific type of genetic analysis.

It should be noted that it is possible to use a specific kit 2 for a specific genetic analysis. Whenever a new type of genetic analysis is to be carried out, a specific kit 2 must be used. Furthermore, the plurality of instruments 3 that is used in combination with the kit 2 in each genetic analysis is substantially the same for each type of genetic analysis.

Preferably, said kit 2 comprises mono-doses of reagents adapted to carry out a specific type of genetic analysis of the sample in an analysis unit 5. Preferably, the reagents inside the kits 2 are ready for use and disposable so as to exclude cross and environmental contaminations. Preferably, a kit 2 comprises a package 21 conformed so as to house the reagents. Each kit 2 comprises an RFID (i.e., Radio Frequency Identification) tag 22 applied for example stably on the package 21 of the kit 2 or inserted inside the package 21 of the kit 2. The RFID tag 22 comprises information data stored therein and representative of a plurality of steps that are required in order to perform the genetic analysis. Preferably, the RFID tag 22 is associated with a microchip and/or a memory in which the information data are stored. Even more preferably, the RFID tag 22 does not need to be powered and comprises a passive RFID antenna. Specifically, the information data are as a function of each kit 2 and comprise for example a text file containing the plurality of steps. The information data can also provide information about the batch and the expiry date of the respective kit 2. By way of example, think of the kit 2 as a package 21 which encloses all the reagents for performing a specific genetic analysis. The RFID tag 22 is applied to the package or is contained therein so that it can be easily read by a suitable reader, for example by means of NFC (i.e., near field communication) technology. Preferably, the RFID tag 22 is stably applied to the outside of the package 21. Even more preferably, the RFID tag is incorporated in the wall of the package 21.

In the following, for brevity's sake, reference will be made in a non-limiting way to a kit 2 as a set of reagents, components and raw materials intended for a single specific application of genetic analysis (for example “kit for the diagnosis of the celiac disease”, “Kit for the virosis of the vine”). In particular, the reagents will be referred to as the set of all reagents necessary for performing the genetic analysis, from step a) of extracting the nucleic acid (DNA and RNA) to step c) of detecting the final result. In the case of specific applications, the reagents may also comprise some accessories necessary for the preparation of the sample (e.g., scalpel, core drill, sharpener, pen for papers, columns for purification).

Preferably, in order to carry out step c) of detecting the final result, each kit 2 comprises mono-doses of reagents for performing genetic analyses with LAMP technology.

Each command and control unit 6 is configured to be able to read the information data contained in the RFID tag 22. In particular, the at least one kit 2 is placed in signal communication with the command and control unit 6 in such a way that the latter can automatically read the information data contained in the RFID tag 22, integrated in the package 21 of the kit 2 or contained therein, and transmit them to the processing unit 16.

The processing unit 16 is configured to receive the information data stored in the RFID tag 22 in order to associate the information data to the plurality of steps so as to display the plurality of steps through the graphic display interface 7. In other words, the processing device 6 is capable of automatically reading the information data stored in the RFID tag 22, so as to associate the respective plurality of steps necessary to perform the genetic analysis with the information data. Furthermore, the processing unit 16 displays the plurality of steps through the graphic display interface 7 so that the user 4 can correctly perform the genetic analysis. Advantageously, the processing unit 16 is able to guide the user 4 step by step during the performance of the genetic analysis through the graphic display interface 7.

The present invention also relates to a method for performing a genetic analysis through a system 100 or an assembly 200 of the type described.

The method comprises a step wherein each command and control unit 6 automatically reads the information data stored in the RFID tag 22 present on the package 21 of the kit 2 or contained therein. The processing unit 16 then displays the plurality of steps through the graphic display interface 7. In accordance with a preferred embodiment, this step is started following a step in which the user 4 activates the processing unit 16 when he identifies himself through the graphic display interface 7, by entering an identification code or by using a badge or by using a radio frequency bracelet. In an alternative embodiment, a power button is provided which is capable of activating the processing unit 16 and the entire assembly 200. Advantageously, the command and control unit 6 automatically recognizes the type of kit 2 by reading the information data contained in the RFID tag 22, and consequently the processing unit 16 recognizes the type of genetic analysis to be carried out. In this way, the processing unit 16 associates the information data with the plurality of steps of the genetic analysis and displays the plurality of steps that the user 4 must carry out for the genetic analysis of at least one sample to be completed on the graphic display interface. Even more advantageously, the processing unit 16, also through the graphic display interface 7, is able to gradually indicate to the user 4 the steps that he must carry out for performing the genetic analysis. It should be noted that a non-specialized user 4 is able to perform a genetic analysis using the assembly 200, following the instructions displayed on the graphic display interface 7.

The method comprises a step wherein the command and control unit 6 processes at least a first signal S1 received by at least one sensor 8 and sends it to the processing unit 16 which generates the first message as a function of the first signal S1. The method therefore comprises a step of displaying the first message through the graphic display interface 7 as a function of the first signal S1. In other words, the processing unit 16 receives at least a first signal S1 which is a function of a physical quantity relative to the genetic analysis, and processes said first signal S1 so as to display a first message through the graphic display interface 7. This message is intended to provide the user 4 with feedback on the steps he is executing, in order to inform him about the presence of any errors in the performance of the genetic analysis. In this way, if an error is detected in a step of the genetic analysis, the user 4 is informed through the first message. In accordance with a preferred embodiment, the processing unit 16, also through the graphic display interface 7, is able to clearly and precisely indicate to the user the subsequent step of the genetic analysis that he will have to carry out. It is worth recalling that the first message also comprises information on the steps that the user 4 must perform in order to carry on in the genetic analysis following an error detected by the processing unit 16 by means of the sensors 8. Advantageously, the user is put in a position in which he is able to repeat the steps in which execution errors have been detected, in order to be able to perform the genetic analysis correctly. It is worth pointing out that a non-specialized user 4 can use the system 100 or the assembly 200 to perform a genetic analysis in a safe and reliable manner thanks to the operating principle of the processing unit 16.

Preferably, the method comprises a step in which the command and control unit 6 processes at least a second signal S2 received by the amplification and optical detection device 31 and sends it to the processing unit 16 which generates the second message as a function of the second signal S2.

In accordance with a preferred embodiment, the method comprises a step in which the system 100 or the assembly 200 recognizes whether the at least a second signal S2 is affected by errors deriving from an incorrect performance of the genetic analysis by the user 4.

The method further comprises a step in which the processing unit 16 displays the second message through the graphic display interface 7. In this way, the user 4 can automatically view the results of the genetic analysis contained in the second message.

In accordance with an embodiment, the method comprises a step in which the processing unit 16 transmits (e.g., via a wired or Wi-Fi internet connection) the information data and/or the first signal S1 and/or the second S2 signal to the remote control unit 10. Advantageously, it is possible to carry out a further monitoring of the genetic analysis at a distance by a remote control unit 10 associated, for example, with a specialized laboratory capable of validating the genetic analysis performed by the non-specialized user 4 by means of the system 100 or the assembly 200.

Advantageously, the remote control system 10 can be associated with a certified central laboratory, equipped with specialized staff, whose task is to remotely monitor the operations carried out in situ by the non-specialized user 4, validating the results of the genetic analysis and ensuring the correct execution thereof.

Advantageously, by using a device 1, system 100 and assembly 200, it is possible for non-specialized staff to carry out genetic analyses in situ or in places other than molecular biology laboratories, ensuring the validation of the results of the genetic analyses through processing unit 16. Furthermore, the device 1, system 100 and assembly 200 allow obtaining field genetic analyses the quality of which is the same as those obtained in the laboratory, as they can be transported easily and in total safety, thanks to the reduced sizes and weight of the device 1. Therefore, a non-specialized user 4 is able to perform genetic analyses with high reproducibility and reliability by means of the device 1, system 100 and assembly 200 for genetic analyses.

Obviously, an expert skilled in the art, for the purpose of satisfying specific, contingent needs, can make numerous modifications to the variants described above, all contained within the scope of protection, as defined by the following claims.

1.-7. (canceled)

8. An assembly for performing in situ genetic analyses, comprising:
a system comprising:
a device conformed so as to be transportable manually by a user, comprising:
a casing defining an internal compartment;
an analysis unit housed in the internal compartment and comprising:
a sample holder compartment accessible by the user and adapted to accommodate at least one sample, the analysis unit being configured to perform a genetic analysis of said at least one sample;
a command and control unit;
at least one sensor selected among an optical, acceleration, temperature, pressure, motion, chemical sensor or a combination thereof, said at least one sensor being configured to detect a first physical quantity relative to said genetic analysis of said at least one sample and to transduce said first physical quantity into a first signal which is indicative of the state of progress of the genetic analysis, said command and control unit being in signal communication with said at least one sensor for receiving said first signal;
a plurality of instruments configured to perform the genetic analysis of said at least one sample, said plurality of instruments comprising an amplification and optical detection device configured to detect at least a second physical quantity relative to said genetic analysis and to transduce said second physical quantity into at least a second signal which is indicative of the outcome of said genetic analysis, said command and control unit being in signal communication with said amplification and optical detection device for receiving said second signal;
said device comprising:
a plurality of said analysis units arranged in said internal compartment, each analysis unit being configured to perform a respective and independent genetic analysis;
said system comprising:
a processing unit, in signal communication with the command and control unit of each analysis unit for receiving the respective first signals and the respective second signals,
a graphic display interface in signal communication with the processing unit,
wherein
the processing unit is configured to display a first message as a function of each first signal through the graphic display interface,
the processing unit is configured to display a second message as a function of each second signal through the graphic display interface;
the assembly further comprising
at least one kit for genetic analyses comprising mono-doses of reagents adapted to carry out a specific type of genetic analysis on the at least one sample in at least one analysis unit, said at least one kit comprising a package conformed so as to house the reagents, said at least one kit further comprising an RFID tag stably applied to said package or contained therein, said RFID tag comprising stored information data representative of a plurality of steps which are necessary to perform said genetic analysis, said information data being as a function of said at least one kit, the at least one kit being placed in signal communication with the command and control unit, each command and control unit being configured to automatically read the information data contained in the RFID tag and transmit them to the processing unit.

9. The assembly according to claim 8, wherein the analysis units are arranged in an array in the internal compartment.

10. The assembly according to claim 8, comprising four analysis units for performing up to four independent genetic analyses.

11. The assembly according to claim 8, wherein each analysis unit comprises a door which can be opened to allow said user to access the sample holder compartment to carry out operations on the sample to analyse.

12. The assembly according to claim 8, wherein the processing unit is in signal communication with a remote control unit positioned externally to said system to transmit each first signal and/or each second signal, said remote control unit being configured to receive and store said first signal and/or said second signal in a database.

13. The assembly according to claim 8, wherein
each kit comprises mono-doses of reagents for performing genetic analyses with LAMP technology;
each amplification and optical detection device comprises a heater for controlling the temperature of the at least one sample during the genetic analysis and an optical and optoelectronic detector for stressing and detecting the fluorescence of the at least one sample.

14. A method for performing a genetic analysis through an assembly according to claim 8, comprising the steps of:
a1) reading said information data stored in said RFID tag and automatically recognizing the type of kit by reading the information data contained in the RFID tag in order to recognize the type of genetic analysis to be carried out, associating the information data with the plurality of steps of the genetic analysis and displaying, through said graphic display interface, the plurality of steps to be carried out by a user for the genetic analysis of at least one sample to be completed;
a2) processing said at least one first signal and displaying said first message through said graphic display interface as a function of the first signal;
a3) processing said at least one second signal and displaying said second message through said graphic display interface as a function of the second signal.
